(19) United States
(12) Patent Application Publication
(10) Pub. No.: US 2010/0068517 A1
(43) Pub. Date: Mar. 18, 2010

(54) ENHANCED SILK PROTEIN MATERIAL HAVING IMPROVED MECHANICAL PERFORMANCE AND METHOD OF FORMING THE SAME

(75) Inventors: Xiang Yang Liu, Singapore (SG); Ning Du, Singapore (SG)

Correspondence Address: OSHA LIANG L.L.P., TWO HOUSTON CENTER, 909 FANNIN, SUITE 3500, HOUSTON, TX, 77010, US

(73) Assignee: NATIONAL UNIVERSITY OF SINGAPORE, Singapore (SG)

(21) Appl. No.: 12/441,114

(22) PCT Filed: Sep. 12, 2007
(86) PCT No.: PCT/SG2007/000307

§ 371 Date: Mar. 30, 2009

Related U.S. Application Data
(60) Provisional application No. 60/843,999, filed on Sep. 12, 2006.

(51) Int. Cl. B32B 1/00; H05F 3/00; C07K 14/435

(52) U.S. Cl. 428/401; 204/165; 530/353

(57) ABSTRACT

The invention provides an enhanced silk fiber rivaling spider silk in mechanical performance, in combination with a very low-cost method for producing it from the usual silkworms. The method provides for the simple application of an electric field which results in an enhancement of over (?) 40% in the strength, and of 200% in the breaking energy with respect to ordinary silkworm silk. The critical elasticity is enhanced to the level of the dragline spider silk. The provided enhanced silk protein material has the same protein primary structure, fiber diameter and length of the customary silk. The method of formation offers the following advantages in comparison to other methods available in the prior art. Industrial scale production can be readily and cost-effectively achieved, given the wide-range availability of silkworms. The provided method relies largely on the present standard production processes of silkworm silk, and hence a low level of investment is required. Since no additional chemicals are required, the provided method is environmentally friendly.

### CROSS REFERENCE

This application claims the benefit of USPTO application No. 60/843,999.

### TECHNICAL FIELD

This invention relates to a protein material, such as protein fiber, providing improved performance, in particular an enhanced silk fiber providing improved mechanical performance and a method of formation of the same.

### BACKGROUND

Natural protein materials such as silk fibers produced by invertebrate species comprise secretions of fibrous proteins. Multiple methods have thus far been devised to improve the mechanical properties of silk protein materials. These attempts suffer from the drawback of being expensive, producing silk protein materials in a form that is inconvenient to use, or in commercially unattractive quantities. Customarily, the prior art emphasizes various reeling protocols of pulling or otherwise forcing the silk fiber from the animal as it is being externally spun into fiber. For example the silkworm will bite and disrupt the fiber growth during reeling, after a certain limited length, making it impossible to obtain a silk fiber of natural length by reeling. Attempts to improve the strength of silk protein materials through gene modification have been of limited success or have been unsuccessful and moreover they are extremely costly.

Spider dragline silk is a high-performance fiber with mechanical properties rivaling the best man-made materials. It can be utilized for making bullet-proof vests, parachutes, collision resistant devices and other high toughness products. However the limited availability of spider silk has brought about an acute market need to produce artificial spider silk. Although much effort, notably including gene modification, has been expended in order to achieve this goal, not much progress has been booked in the current extensive prior art. An alternative option worth pursuing which will be taught in this invention is to convert ordinary or natural silkworm silk into an enhanced silk protein material approaching the performance of spider silk. It is worth noting that from the point of view of application, that the enhanced silkworm silk will be superior to spider silk as regards shelf-life or storage stability, because spider silk is biodegradable much more rapidly than silkworm silk.

What is mostly needed in the prior art is an enhanced silk protein material that is fairly inexpensive to produce, is capable of massive industrial-scale production, requires modest and easy-to-construct apparatus, and avoids the limitations of the prior art. Specifically the much needed enhanced silk protein material should offer superior mechanical performance without the need of reeling, while leaving all other properties of natural silk fibers unaffected. Specifically it should provide for silk fibers of natural length without compromising its enhanced mechanical properties. Such a product and method of fabrication does not exist in the prior art, and it is the goal of the present invention.

### REFERENCES

1) http://www.uwyo.edu/news/showrelease.asp?id=8959
2) http://www.nexiabiotech.com/en/01_tech/01-bst.php
3) Chen, X. et al., Chinese patent, 200510024440.1 (2005)
4) Shao, Z. et al., Chinese patent, 200510024438.4 (2005)
5) Xu, M. & Lewis, R. V. (1990) Proc. Natl. Acad. Sci. USA 87, 7120-7124.
6) Kohler, T. & Vollrath, F. (1995) J. Exp. Zool. 271, 1-17.
7) Shao, Z. & Vollrath, F. (2002) Nature 418, 741.
8) Lazaris, A. et al., (2002) Science, 295, 472-476.
9) Du, N., Liu, X. Y., Narayanan, J., Li, L., Lim, M. & Li, D. (2006) Biophys. J. 91, 4528-4535.
10) Philip M. Cunniff. et al, Mechanical properties of Major ampulate gland silkfibers extracted from Nephila clavipes spiders. In silk polymers: materials science and biotechnology (eds. David Kaplan, W. Wade Adams, Barry Farmer, and Christopher Viney). ACS symposium Series 544. (1993) pp. 235-251
11) Fritz Vollrath & David P. Knight, Liquid crystalline spinning of spider silk. Nature, 410, 541-548, 2001.

### SUMMARY

In an embodiment of the present invention there is provided an enhanced protein material having a dimension longer than 20 mm, the enhanced protein material having an enhanced mechanical performance that is higher than a natural mechanical performance of a natural silk fiber, wherein the natural silk fiber is produced naturally by an animal of an invertebrate species, in an absence of an intervention, wherein the enhanced protein material is not a regenerated silkworm-protein material, and wherein the invertebrate species is not a spider species.

In another aspect there is provided an enhanced protein material having a dimension longer than 20 mm, the enhanced protein material having a higher mechanical performance than a natural mechanical performance of a natural silk fiber, the enhanced protein material having a higher crystallite alignment degree than a natural crystallite alignment degree of the natural silk fiber, wherein the natural silk fiber is produced naturally by an animal of an invertebrate species in an absence of an intervention, wherein the invertebrate species is not a spider species.

In another aspect the mechanical performance may be at least one selected from the group comprising yield stress, yield strain, breaking stress, breaking strain, breaking energy and elastic modulus. In another aspect the protein material may be a protein silk fiber. The yield stress of the protein fiber may be more than 140 MPa or more than 150 MPa. The yield strain of the protein fiber may be more than 1.7% or more than 1.8%. The breaking stress of the protein fiber may be more than 498 MPa or more than 505 MPa. The breaking strain of the protein fiber may be more than 16% or more than 17%. The breaking energy of the protein fiber may be more than 40 kJ/kg or more than 50 kJ/kg. The elastic modulus of the protein fiber may be more than 9 GPa or more than 10 GPa. The disclosed protein fiber may comprise substantially the same primary protein structure as a natural protein fiber. The protein fiber may have a diameter substantially equal to a natural diameter of a natural protein fiber. The protein fiber may respond to environmental conditions comprising a temperature and a humidity in a way that does not differ substantially from a natural protein silk fiber.

The protein silk fiber may have a degree of crystallite alignment that is higher relative to fiber produced from an animal that has not been subjected to the stimulus. The comprehensive orientation function <ƒ> of the protein fiber may be at least larger than 0.84 or larger than 0.89.

In another aspect there is provided a method of formation of an enhanced protein material comprising the steps of:

a) taking an animal of an invertebrate species that is able to extrude a protein material,
b) applying a stimulus to the animal, wherein the stimulus excludes reeling,
the stimulus enhancing a mechanical performance of a natural protein material,
the natural protein material being the protein material extruded by the animal in an absence of the stimulus,
and
c) collecting the enhanced protein material extruded by the animal.

In another aspect the stimulus as disclosed herein may be an electromagnetic field such as an electric field or an optical stimulus such as light radiation. The electric field may be an alternating electric field or a constant electric field. The peak-to-peak strength of the alternating electric field may be from about more than 0 V/cm to about 2000 V/cm or from about more than 0 V/cm to about 600 V/cm. The frequency of the electric field may be from 0 MHz to about 2 MHz or from 0 MHz to about 1 MHz or from 0 MHz to about 5000 MHz. In another embodiment, the stimulus may be any stimulating means that is able to improve the crystallite alignment degree and mechanical performance of a natural protein fiber produced by an animal.

In another aspect there is provided a method of formation of an enhanced protein material comprising the steps of:

a) taking an animal of an invertebrate species that is able to extrude a protein material,
b) applying a stimulus to the animal, wherein the stimulus excludes reeling,
the stimulus enhancing a mechanical performance of a natural protein material,
the natural protein material being the protein material extruded by the animal in an absence of the stimulus, and
c) collecting the enhanced protein material extruded by the animal.

The invertebrate species may be a silkworm species. The silkworm species may be selected from a group comprising Bombyx mori, Philosamia Cynthia and Telea Polyphemus.

In another aspect there is provided a method of formation of an enhanced silk fiber, the method comprising the steps of:

a) taking one or more silkworms,
b) preparing an apparatus for applying an alternating electric field to the one or more silkworms,
the alternating electric field having an alternating electric field strength is in a range 0-600 V/cm and an alternating electric field frequency is in a range 0-5000 Hz, the apparatus comprising a container,
the container being suitably partitioned for placing the one or more silk worms such that
the alternating electric field remains unaffected by the container,
c) placing the one or more silkworms in the partitioned container,
d) activating the alternating electric field when the one or more silkworms begin a spinning process, thereby bringing about an enhancement of a mechanical performance of a natural silk fiber, the natural silk fiber being extruded by the one or more silk worms in an absence of the alternating electric field,
the mechanical performance comprising one or more of: a yield stress, a yield strain, breaking stress, a breaking strain, a breaking energy and an elastic modulus,
e) maintaining the alternating electric field until completion of the spinning process, and
f) collecting the enhanced silk fiber extruded by the one or more silkworms.

In another aspect, there is provided the use of the apparatus as defined above. In another aspect, there is provided a composition comprising the protein fiber as disclosed above. In another aspect, there is provided a mixture comprising the protein fiber as disclosed above. In another aspect, there is provided a product comprising the composition and/or the mixture as defined above.

### DEFINITIONS

The following words and terms used herein shall have the meaning indicated.

In the best mode for carrying out the invention the enhanced protein material is a protein silk fiber or silk fiber, for which reason these two terms are used interchangeably.

The term ‘natural protein material’ and more specifically the term ‘natural protein fiber’ that in the disclosed embodiment is a silk fiber, is to be interpreted to include any fiber material that is spun from an animal capable of producing protein fiber in the customary production environment, meaning not in a specific laboratory environment. In the context of the disclosed embodiment the “natural protein material” is denoted as “cocoon silk”, or “cocoon”. The term “control protein material” and more specifically the term “control protein fiber” produced by a the aforementioned animal, or “control silk fiber”, denotes a silk fiber produced by a silkworm in a specific laboratory environment in which the animal has not been subjected to a stimulus to enhance the mechanical performance of the fiber, all other experimental conditions remaining the same.

The word “enhanced” when referring to a “protein material” or more specifically “protein fiber” or a “silk fiber” refers to material or fibers that have higher mechanical properties relative to natural material or natural fibers or control material or control fibers as defined above.

In one disclosed embodiment of the invention, the stimulus is an electric field, that can be constant or alternating. In that case, the enhanced protein fiber refers to silk fiber produced when silkworms are subjected to the electric field and also a certain amount of room light. The control protein fiber refers to silk fiber produced when the field is switched off, all other conditions remaining the same. That means that the silkworms producing the control protein fiber are exposed to the same amount of room light as the silkworms producing the enhanced silk fiber.

In another disclosed embodiment of the invention, the stimulus is optical radiation generated by room light. In that case, the enhanced protein fiber refers to silk fiber produced when silkworms are subjected to the full amount of available room light. The control protein fiber refers to silk fiber produced when the room light is switched off, meaning that the silkworms are kept in almost total darkness.

The mechanical performance of protein fibers is expressed in terms of rheological quantities as defined in this section as well as in the section on the detailed description below. In the state of the art the employed rheological quantities have specific meanings which, to avoid confusion, should not be used loosely or interchangeably. The “strength” of the fiber is measured by the “breaking stress”, that is the amount of stress which causes the fiber to break; this is expressed in units of pressure or Pascal, equivalent to one newton per square meter (Pa=N/m²). The “elongation” of the fiber is measured by the “breaking strain”, that is the amount of strain exhibited by the fiber at the breaking point, and it is expressed as a percentage of the original length of the fiber prior to elongation. The “toughness” of the fiber is measured by the “breaking energy”, that is the amount of work needed to break the fiber; this is expressed in units of energy per unit mass of the fiber or kilo Joule per kilogram (kJ/kg). The elastic modulus or Young's modulus measures elasticity or stiffness in units of pressure (Pa).

The structural properties of protein fibers are expressed in terms of “comprehensive orientation functions” specified at length in the detailed description. Protein fibers and specifically silk fibers comprise amorphous and crystalline regions. An orientation function ƒ measures the alignment degree among the crystallites within a single fiber. A comprehensive orientation function <ƒ> is defined as a group average of the crystallite orientation functions ƒ. The enhanced protein fibers are characterized by a comprehensive orientation function <ƒₑ>, whereas the natural or control protein fibers are characterized by a comprehensive orientation function <ƒₙ>.

As explained in the detailed description, in one embodiment, alternating and constant electric fields are employed. In the case of an alternating electric field, the measurement of the corresponding voltage, which is a variable quantity, is referred to as a “peak-to-peak voltage”, meaning the maximum value of the voltage. Accordingly, the measurement of the associated electric field strength is referred to as a “peak-to-peak electric field strength”. In the case of a constant electric field the disclosed measurements of the voltage and the electric field strength follow the standard well known expressions and conventions. In both cases the voltage is expressed in units of volts (V) and the electric field in volts divided by distance (V/cm).

In the case of an alternating field, the frequency is expressed following the standard conventions in units of hertz (H). The disclosed frequency ranges are approximate. However, when a disclosed frequency range starts from 0, it should be understood that the zero frequency value is meant to be exact, since the associated electric field is not alternating but constant.

### BRIEF DESCRIPTION OF THE DRAWINGS

One embodiment of the present invention is illustrated by the following drawings:

FIG. 1 is a graphical illustration of the superior mechanical properties of an enhanced protein material as compared with protein materials of the prior art;

FIG. 2 is a schematic diagram of a production process of the enhanced protein material;

FIG. 3 is an illustration of an effect of an electric field applied to a silking silkworm;

FIG. 4 is a schematic illustration of an apparatus used in a disclosed embodiment;

FIG. 5 illustrates mechanical properties by a stress vs strain curve;

FIG. 6 shows contour lines of a breaking stress of the enhanced protein material;

FIG. 7 shows contour lines of a breaking strain of the enhanced protein material;

FIG. 8 is a stress vs strain curve of the enhanced protein material and a control silk;

FIG. 9 is the stress vs strain curve of the enhanced protein material, a control silk and a spider silk; and

FIG. 10 compares the enhanced protein material with other silk and artificial polymer materials with reference to a breaking energy.

### DETAILED DESCRIPTION

### Introduction

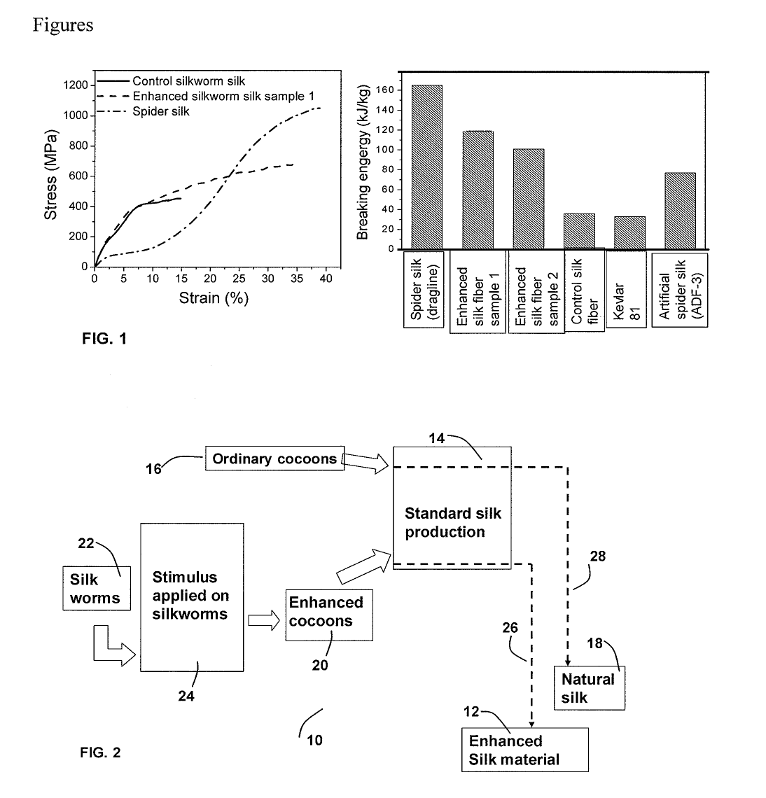

The disclosed exemplary process for producing an enhanced silk protein material by applying a stimulus to an animal of an invertebrate species, are described hereinafter with reference to FIG. 1 in FIGS. 2-10. The present embodiment refers to the enhanced silk protein material produced from silkworms, and serves the purpose of illustration only, in no way limiting the scope of the invention. A cocoon silk is a commonly farmed silk when silkworms are fed mulberry leaves. In the present embodiment the stimulus is an electromagnetic field and more specifically an electric field. The control silk is defined as a protein material obtained under identical laboratory conditions as the enhanced protein material, with the only exception that the animal is not subjected to any stimuli.

Silkworms used in the embodiment, in connection with the control silk and the enhanced protein material, are fed a substitute of mulberry leaves. Because the mechanical properties of the cocoon silk and the control silk are very similar, the cocoon silk and the control silk are denoted as “natural silks”. Hardly any difference is observed between the cocoon silk obtained from silkworms fed mulberry leaves and the control silk obtained from silkworms fed mulberry leaf substitute.

### One Embodiment of the Invention

FIG. 2 shows how a production process 10 of the enhanced protein material that is a silk fiber 12 can be inserted in a standard production process 14 of ordinary cocoons 16, that means cocoons comprising natural silk 18, in a routine and cost-effective way. To produce the enhanced protein material 12, silkworms 22 are subjected to the stimulus 24. The resulting enhanced cocoons 20 comprising the enhanced protein material 12, are handled with the same standard production process 14 as the ordinary cocoons 16 comprising the natural silk 18. Therefore the enhanced protein material 12 is obtained 26 upon completion of the same standard production process 14 by which the natural silk 18 is routinely produced 28.

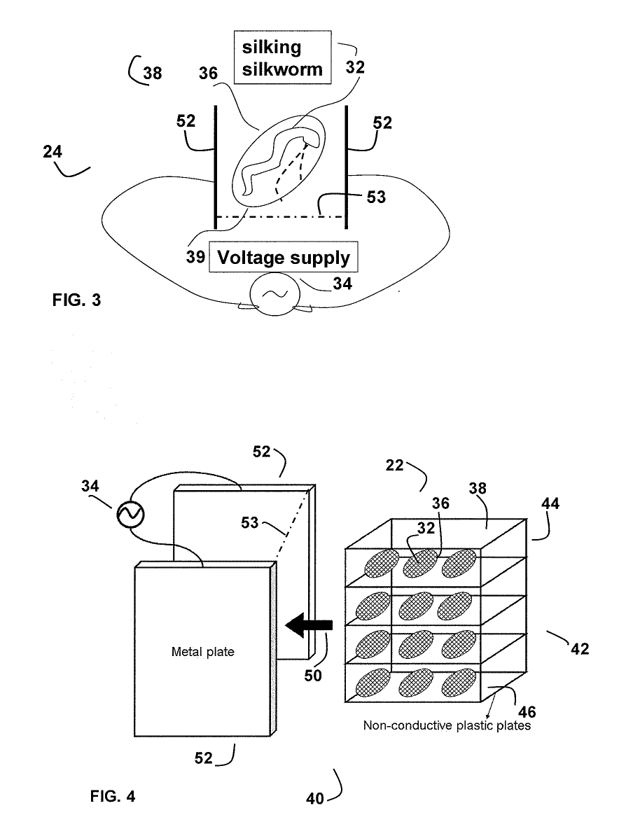

FIG. 3 illustrates the effect of the stimulus 24 on a silking silkworm 32, in the present embodiment. One silkworm 32 is placed in a compartment 36 belonging to an array 38 of compartments. The stimulus 24, in the embodiment being the electric field associated with a voltage 34, is applied across the array 38 of compartments that is sandwiched between two metal plates 52, separated by a separation distance 53. The electric field can be an alternating field and it can have a frequency. The frequency can be equal to zero, in which case the electric field is a constant electric field. The separation distance 53 should be small enough to ensure uniform distribution of the electric field applied on the array 38 of compartments, resulting in equal responsiveness of each silkworm 32 to the electric field. On the other hand the separation distance 53 should be large enough to ensure proper ventilation of the silkworm 32. An exemplary suitable separation distance 53 is at least 1.5 cm (i.e. the approximate diameter of the silk worm body) and more preferably about 5 cm to about 6 cm.

FIG. 4 illustrates an apparatus 40 used for subjecting the silkworms 22 to the electric field produced by the voltage 34. The apparatus 40 comprises a container 42 with a spatial partitioned arrangement 44 comprising the array 38 of compartments, accommodating one silkworm 32 in each compartment 36. The container 42 is made of non-conductive material 46, so as not to interfere with the applied electric field, and it is inserted 50 between surfaces that are the conducting metal plates 52 connected to the supply of the voltage 34.

In FIGS. 3 and 4, the separation distance 53 between the plates 52 is larger than 1.5 cm, whereas an optimal value is in a range 5-6 cm. In the present embodiment the dimensions of the metal plates 52 are about 20×20 cm, and the separation distance 53 is 5.5 cm. The applied voltage 34 depends on the separation distance 53. The peak-to-peak strength E of the electric field can be taken in a range 0<E<2000 V/cm, and its frequency can vary from 0 to 2 MHz. In the present embodiment the electric field strength is in a range 0<E<600 V/cm, and its frequency varies from 0 to 5000 Hz.

Referring again to FIG. 3, the silkworm 32 usually begins to spin a cocoon 39 when it is 3-4 weeks old, at which stage the stimulus 24 can be applied. In FIG. 2, the stimulus 24, being the electric field, is turned on the silkworms 22, and it is maintained until completion of a spinning process which takes usually 2-3 days. The enhanced protein material 12 is collected 26, and subsequently it may be further processed by an additional step of post-stretching. This step comprises stretching the enhanced protein material by a fraction, for example by about 10%, maintaining it in a stretched condition for a short period of time, for example 5 min, and allowing it to relax.

### Processing of the Enhanced Protein Material

The protein material that is a silk fiber comprises two proteins, serisin and fibroin. The former is soluble in hot water, while the latter is not. A degumming treatment, such as those known in the art, is used to separate the fibroin strands the unwanted globular serisin by using a solution comprising hot water and soap.

### Mechanical Performance of the Enhanced Silk Protein Material

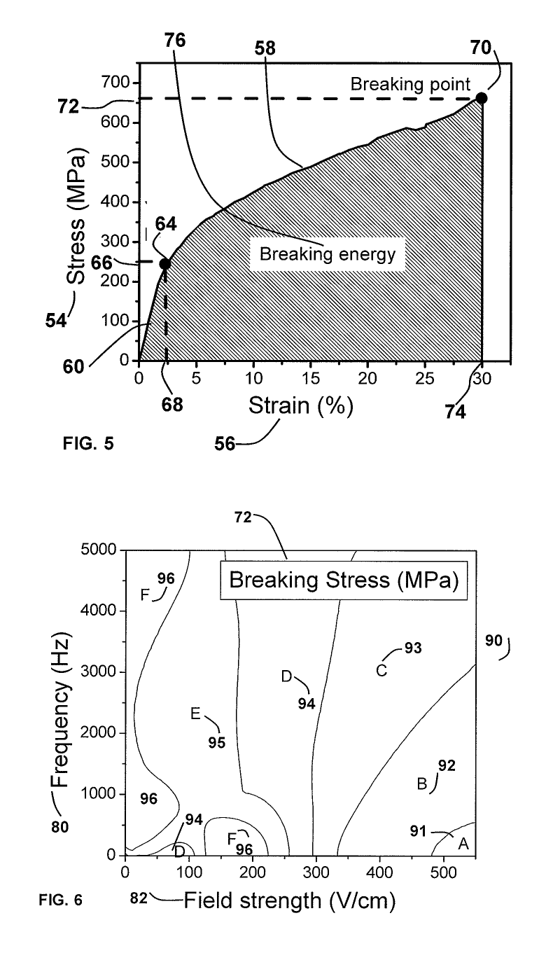

FIG. 5 is a stress 54 vs strain 56 curve 58 illustrating the mechanical properties of a silk fiber. A first part 60 of the curve 58 is linear. The curve starts to deviate from the first linear part 60 at a yield point 64, thus showing a yield stress 66 and a yield strain 68. The slope of the first linear part 60 is Young's elastic modulus (elastic modulus or Young's modulus) defined as the yield stress 66 divided by the yield strain 68. The elastic modulus measures elasticity or stiffness. The silk fiber breaks at a breaking point 70, thus showing a breaking stress 72 that measures strength, and a breaking strain 74 that measures elongation. A breaking energy 76 is an amount of work needed to break the silk fiber, it is equal to an area under the stress 54 vs strain 56 curve 58, and it measures toughness.

### Method of Measurement of Mechanical Properties

FIG. 2 shows the enhanced protein material that is a silk fiber 12 after collection 26, and the natural silks 18 (that is, the control silk or the cocoon silk) after collection 28. An amount of 20-30 fibers are randomly selected from each of 5-10 ordinary cocoons 16, amounting to an ordinary fiber collection of a total of 100-230 natural cocoon silk fibers 18. An amount of 20-30 fibers are randomly selected from each of 5-10 control silk cocoons at zero electric field, amounting to a control fiber collection of a total of 100-230 natural control silk fibers. For each electric field strength and frequency, an amount of 20-30 fibers are randomly selected from each of 5-10 enhanced cocoons 20, amounting to an enhanced fiber collection of a total of 100-230 enhanced silk fibers.

The mechanical properties comprising the yield stress, the yield strain, the breaking stress, the breaking strain, the breaking energy and the elastic modulus, are tested at a room temperature 22-24 C and a humidity 65%-72%. Their experimental values are obtained by averaging over the measurements carried out independently on the ordinary fiber collection; the control fiber collection; and the enhanced fiber collection for each pair of electric field strength and frequency. An Instron MicroTester is employed to measure the force-extension characteristics of the silk fibers. Suitable parameters for the measurements are by way of illustration as follows. A fiber specimen with a gauge length of 20 mm is fixed between two hooks of the instrument, with a measured error of 0.1 mm. The specimen is stretched until it breaks and the strain rate is 50% per minute.

### Definition of the Electric Field as the Stimulus

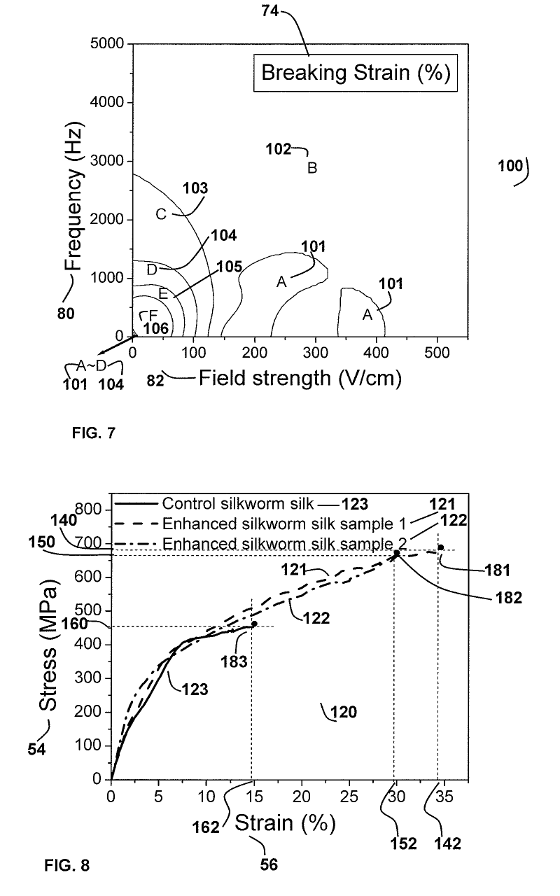

In FIGS. 6 and 7, the applied stimulus is the electric field that can have a zero frequency (constant electric field), or a nonzero frequency (alternating electric field). In FIGS. 3 and 4 we can see that, in either case, the electric field depends on the applied voltage 34 and the separation distance 53 between the plates 52. In the case of an alternating electric field, the term “voltage” 34 refers to a peak-to-peak voltage, and hence the term “electric field strength” refers to a peak-to-peak strength, as indicated above. For example, for a nonzero frequency the peak-to-peak electric field strength is 545 V/cm when the separation distance 53 is fixed at 5.5 cm and the peak-to-peak voltage 34 is fixed at 3000 V. When the frequency becomes adjusted to zero, the resulting constant electric field is 545 V/cm, while the separation distance 53 remains 5.5 cm and the constant voltage 34 is 3000 V/cm.

### Results on Enhanced Mechanical Properties

As an illustration, in the contour plots of FIGS. 6 and 7 many pairs of electric field intensities and frequencies are used for obtaining the breaking stress 72 and the breaking strain 74. The adopted separation distance 53 (shown in FIGS. 3 and 4) used for the measurements is 5.5 cm. FIG. 6 is a contour graph of the breaking stress 72 on a plot of the electric field frequency 80 vs the electric field strength 82. FIG. 7 is a contour graph of the breaking strain 74 on a plot of the electric field frequency 80 vs the electric field strength 82.

In FIGS. 6 and 7, the breaking stress 72 and the breaking strain 74 vary strongly with the electric field strength 82 and the frequency 80. In FIG. 6 the regions A-F 90 mark the following ranges of the breaking stress 72 in MPa: A 91 300-350; B 92 350-400; C 93 400-450; D 94 450-500; E 95 500-550; F 96 more than 550. In FIG. 7 the regions A-F 100 mark the following ranges of the breaking strain 74 in percent: A 101 less than 16.5%; B 102 16.5%-19.0%; C 103 19.0%-21.5%; D 104 21.5%-24.0%; E 105 24.0%-26.5%; F 106 more than 26.50.

FIG. 8 is a comparison of the stress 54 vs strain 56 curves 120 for a sample 1 121 of the enhanced protein material that is a silk fiber, a sample 2 122 of the enhanced silk fiber, and the control silk 123. In FIG. 8 the sample 1 121 is characterized by a sample 1 breaking point 181, that is determined by a sample 1 breaking stress 140 and a sample 1 breaking strain 142. The sample 2 122 is characterized by a sample 2 breaking point 182, that is determined by a sample 2 breaking stress 150 and a sample 2 breaking strain 152. The control silk 123 is characterized by a control breaking point 183, that is determined by a control breaking stress 160 and a control breaking strain 162. In FIG. 6 the ranges D 94, E 95 and F 96 are associated with the breaking stress 72 that is higher than the control breaking stress 160 of FIG. 8, that is equal to 468 MPa.

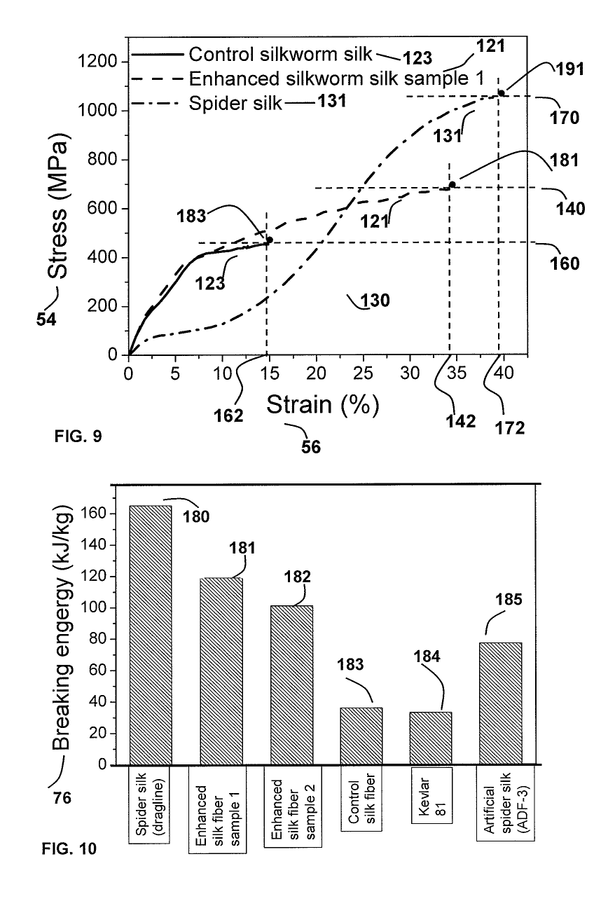

FIG. 9 is a comparison of the stress 54 vs strain 56 curves 130 of the sample 1 121 of the enhanced silk fiber, the control silk 123 and a spider silk 131. The spider silk 131 is characterized by a spider silk breaking point 191, that is determined by a spider silk breaking stress 170 and a spider silk breaking strain 172. The sample 1 breaking point 181, the sample 1 breaking stress 140, the sample 1 breaking strain 142, the control breaking point 183, the control breaking stress 160 and the control braking strain 162 are also shown in FIG. 9. In FIG. 7 all of the ranges A-F 100 are associated with the breaking stress 74 that is higher than the control breaking strain 162 of FIG. 9, that is equal to 14.8%.

FIG. 10 compares the breaking energy 76, as defined in FIG. 5, of the enhanced silk fiber with various silk and synthetic fibers. Only a spider silk breaking energy 180 equal to 165 kJ/kg is higher than a sample 1 breaking energy 181 equal to 124 kJ/kg, and a sample 2 breaking energy 182 equal to 101 kJ/kg. Whereas in FIG. 10, a Kevlar 81 breaking energy 184 equal to 33 kJ/kg, and an artificial spider silk breaking energy 185 equal to 77 kJ/kg (ADF-sample 3), as well as a control silk breaking energy 183 equal to 33 kJ/kg, are markedly inferior to the breaking energies of the enhanced silk fibers.

The mechanical properties, i.e. the breaking stress, the breaking strain, the breaking energy, the yield stress, the yield strain and Young's modulus, of the enhanced silk fiber samples 1 and 2, the control silk and the cocoon silk are summarized in Table 1. The quantities referring to the enhanced silk samples 1 and 2 appear in boldface. Fractional enhancements of the enhanced silk fibers with respect to the control silk appear in italics in square brackets below the corresponding quantities. The mechanical properties of the enhanced silk fibers of the embodiment are compared with the corresponding properties of the spider silk, a spider dragline silk (Araneus), a recombinant spider silk comprising protein ADF-3, and Kevlar, that is a synthetic polymer fiber.

TABLE 1

| | Breaking | Breaking | Breaking | Yield | Yield | Young's |
|---|---|---|---|---|---|---|
| | stress | strain | energy | stress | strain | modulus |
| Material | (MPa) | (%) | (kJ/kg) | (MPa) | (%) | (GPa) |
| Cocoon silk | 491 | 14.9 | 37 | 138 | 1.6 | 8.6 |
| Control silk | 468 | 14.8 | 36 | 137 | 1.6 | 8.5 |
| Enhanced silk | 662 | 29.8 | 101 | 233 | 1.9 | 12.2 |
| sample 2 | [42%] | [101%] | [182%] | [70%] | [19%] | [44%] |
| Enhanced silk | 682 | 34.4 | 124 | 158 | 1.8 | 9 |
| sample 1 | [46%] | [132%] | [244%] | [15%] | [13%] | [6%] |
| Kevlar 29^{a} | 2800 | 3.5 | — | — | — | 62 |
| Kevlar 49^{a} | 2800 | 2.5 | — | — | — | 124 |
| Kevlar 81^{b} | 3600 | 5 | 33 | — | — | 90 |
| Spider silk^{b} | 1200 | 39 | 165 | 150 | — | 7.9 |
| ADF-3 silk | 230 | 59.6 | 107 | — | — | — |
| sample 1^{c} | | | | | | |
| ADF-3 silk | 270 | 43.4 | 101 | — | — | — |
| sample 2^{c} | | | | | | |
| ADF-3 silk | 210 | 45.0 | 77 | — | — | — |
| sample 3^{c} | | | | | | |
| Araneus, spider | 800-1300 | 19-30 | 72-155 | — | — | — |
| dragline silk^{c} | | | | | | |

^{a}Philip M. Cunniff. et al, Mechanical properties of Major ampulate gland silk fibers extracted from Nephila clavipes spiders. In silk polymers: materials science and biotechnology (eds. David Kaplan, W. Wade Adams, Barry Farmer, and Christopher Viney). ACS symposium Series 544. (1993) pp.235-251
^{b}Fritz Vollrath & David P. Knight, Liquid crystalline spinning of spider silk. Nature, 410, 541-548, (2001).
^{c}A. Lazaris, et al. Spider Silk Fibers Spun from Soluble Recombinant Silk Produced in Mammalian Cells, Science. 295, 472 (2002).

It will be appreciated from the above data, that when the electric field was applied to the silkworms, the mechanical performance of the enhanced silk increased relative to the control group. The results of Table 1 show that the enhanced silk fiber of sample 1 has a breaking energy equal to 124 kJ/kg, that is a factor 3.3 higher than the control or cocoon silk, and hence it is tougher than all of the tabulated materials except for the extruded spider silk fibers. The enhanced silk fiber sample 2 has a breaking energy equal to 101 kJ/kg, that is a factor 2.7 higher than the control or cocoon silk. Kevlar is about four times stronger than the enhanced silk samples 1 and 2 (breaking stress larger by a factor about 4), and three times stronger than the spider silk (breaking stress larger by a factor 3). However both of the enhanced silk samples and spider silk are 3-4 times tougher than Kevlar (breaking energy larger by a factor about 3-4) because they are 6-14 times more extendable (breaking strain larger by a factor about 6-14).

### Structure of the Enhanced Protein Material

The enhanced protein material, which in the embodiment is the enhanced silk fiber, has substantially the same primary protein structure, because the protein is already formed inside the silkworm before application of the stimulus, being the electric field. An analogous argument holds for the diameter of the enhanced silk fiber being substantially equal to the diameter of the control silk fiber.

In general, silk comprises amorphous and ordered protein regions. The ordered protein regions comprise protein beta sheets as well as crystallites comprising protein beta sheets. The crystallites are investigated by X-ray diffraction. The wide angle X-ray scattering (WAXS) was used to measure the size and alignment of crystallites and crystallinity of silk fiber. The small angle X-ray scattering (SAXS) was used to measure the inter-crystallite distance.

The wide angle X-ray scattering (WAXS) patterns of a bundle of 800 silkworm silk fibers were collected using a Bruker GADDS X-ray diffractometer with beam size 0.5 mm. The radiation wavelength used was 1.5418 Å for Cu Ka. The sample-to-detector distance was 6 cm and the exposure time was 30 min.

The small angle X-ray scattering (SAXS) experiments were performed with a Bruker NanoSTAR small angle X-ray scattering system. The generator was operated at 40 kV and 35 mA. A double pinhole system creates an X-ray spot with diameter 200 mm on the silk and the detector has a spatial resolution of 10 mm. The detector-to-sample distance is 107 cm. The sample chamber was placed under vacuum to eliminate scattering of air. The silk fibers were placed parallel to one another between two thin glass plates of thickness 0.1 mm, which were glued together on the edges to prevent water evaporation from the silk fiber in vacuum.

The crystal lattice parameters defining a crystallite unit cell can be determined from the outcome. The crystallites of the enhanced silk fiber sample 2 and the control silk were investigated by X-ray diffraction. These crystallites have an orthorhombic unit cell. The crystallite dimensions lₐ, l_{b} and l_{c}, as determined by X-ray diffraction, are tabulated in Table 2. The longest dimensions of the crystallites, l_{c}, have various orientations with respect to the fiber axes. The distances among the crystallites as well as the angles between the various crystallites and the fiber axes can be determined by X-ray diffraction. An orientation function ƒ measures a degree of alignment among the crystallites:

$$f = \frac{1}{2} ( 3 〈 {cos}^{2} ϕ 〉 - 1 )$$

When all the long dimensions l_{c} of the crystallites are aligned parallel to the silk fiber longitudinal axis, ƒ=1; when all are aligned perpendicular to that axis, ƒ=−½. When the long dimensions l_{c} are completely randomly oriented, ƒ=0. A comprehensive orientation function <ƒ> is an average of individual orientation functions f over several (about 800) silk fibers. In Table 2, the comprehensive orientation function <ƒₙ> for the control silk fibers is 0.84. The comprehensive orientation function <ƒₑ> for the enhanced silk fibers is 0.9. Crystallinity is a volume fraction of the crystallites in the silk protein material. The crystallinities of the control and enhanced silk fibers equal 27, and do not differ at a detectable level. The distances among the crystallites of the enhanced silk sample 2 and control silk are also tabulated in Table 2. The breaking stress and breaking strain for the enhanced silk sample 2 and the control silk (see Table 1) are also included in Table 2 for completeness.

We observe that the value of the breaking stress increases from the control silk to the enhanced silk fiber. Considering that this increase is accompanied by an increase of the comprehensive orientation function <ƒ> from 0.84 to 0.90, we conclude that the increase in the breaking stress and breaking strain can be attributed to the increase of the degree of crystallite alignment in the enhanced silk fiber.

TABLE 2

| | | Comprehensive | Crystallite size | | | Inter- | Breaking | Breaking |
|---|---|---|---|---|---|---|---|---|
| Silk | Crystallinity | orientation | (nm) | | | crystallite | stress | strain |
| material | (%) | function <f> | lₐ | l_{b} | l_{c} | distance (nm) | (MPa) | (%) |
| Control | 27 | 0.84 | 1.6 | 4.8 | 20.4 | 4.3 | 468 | 14.8 |
| Enhanced | 27 | 0.9 | 1.6 | 5.2 | 20.2 | 4.9 | 662 | 29.8 |
| sample 2 | | | | | | | | |

### Optical Stimuli

In another disclosed embodiment of the invention, the stimulus is optical radiation generated by room light. As explained earlier in the section on definitions, the definitions of “enhanced protein fiber” and “control protein fiber” differ from the corresponding definitions employed in the previous embodiment.

Experiments were carried out to determine the influence of room light on the mechanical properties of silkworm silk. The enhanced protein fiber refers to silk fiber produced when silkworms are subjected to the full amount of available room light. The control protein fiber refers to silk fiber produced in a “dark environment” (hereafter “control protein fiber”), created by putting the silkworms in a box in which light cannot pass through. The group of silkworms are exposed fully to room light by placing the silkworms next to the box on the table where the room light could be directly radiated on these silkworms. Such radiation was continued until the silkworms had finished making cocoons (3 days). The mechanical properties of the silk obtained in these two different conditions are shown in Table 3 below:

TABLE 3

| | Breaking stress | Breaking strain | Breaking energy |
|---|---|---|---|
| Material | (MPa) | (%) | (kJ/kg) |
| Enhanced protein | 489 | 16.7 | 38 |
| fiber with radiation | | | |
| of light | | | |
| Control protein | 409 | 15.4 | 30 |
| fiber in dark | | | |
| environment | | | |

It is shown from the table above that the room light stimulates the silkworms to produce stronger silk as indicated from the increase of breaking stress of the silk from 409 MPa to 489 MPa when the silkworms are subjected to room light radiation. It can also be seen from the table above that there appears to be a slight improvement in the breaking strain of the silk produced by the silkworms that are subjected to room light radiation.

### Applications

The disclosed process provides a method for producing protein material and specifically silk protein fiber that has enhanced mechanical performance relative to natural cocoon silk fibers and control fibers, produced by animals that are not subjected to the stimuli. The disclosed process therefore provides a relatively simple and inexpensive method to produce protein material such as silk fiber while leaving the natural length of the silk fiber unaffected. Hence, the disclosed process can readily be produced on a commercial scale.

The disclosed process does not require the use of any reeling protocols of pulling or otherwise forcing the silk fiber from the animal as it is being spun into fiber. Hence, in the disclosed process, it is possible to produce a fiber of enhanced mechanical performance and increased length relative to other prior art fibers.

The disclosed process does not require the use of gene modification to improve the strength of the fiber and hence is less costly. The disclosed process provides an enhanced silk from silkworms that, due to its enhanced mechanical performance, may be used as a substitute for other naturally produced fibers such as spider dragline silk.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. An enhanced protein material having a dimension longer than 20 mm, the enhanced protein material having an enhanced mechanical performance that is higher than a natural mechanical performance of a natural silk fiber,
wherein the natural silk fiber is produced naturally by an animal of an invertebrate species, in an absence of an intervention,
wherein the enhanced protein material is not a regenerated silkworm-protein material, and
wherein the invertebrate species is not a spider species.

2-27. (canceled)

28. The enhanced protein material of claim 1, wherein the dimension is longer than 10 cm.

29. (canceled)

30. (canceled)

31. The enhanced protein material of claims 1, wherein the natural mechanical performance comprises at least one of a natural yield stress, a natural yield strain, a natural breaking stress, a natural breaking strain, a natural breaking energy and a natural elastic modulus, and wherein the higher mechanical performance comprises at least one of: a higher yield stress than the natural yield stress, a higher yield strain than the natural yield strain, a higher breaking stress than the natural breaking stress, a higher breaking strain than the natural breaking strain, a higher breaking energy than the natural breaking energy and a higher elastic modulus than the natural elastic modulus.

32. The enhanced protein material of claim 1, wherein the enhanced protein material is an enhanced silk fiber extruded by the animal of the invertebrate species, wherein the invertebrate species is a silkworm species selected from a group comprising Bombyx mori, Philosamia Cynthia and Telea Polyphemus.

33. (canceled)

34. The enhanced protein material of claim 31, wherein the high mechanical performance is at least one of a higher yield stress is more than 140 MPa, a higher strain that is more than 1.7%, a higher breaking stress that is more than 498 MPa, a higher breaking strain that is more than 16%, a higher breaking energy that is more than 40 kJ/kg, or a higher breaking elastic modulus that is more than 9 GPa.

35-45. (canceled)

46. The enhanced protein material of claim 1, wherein the enhanced protein material has a higher crystallite alignment degree than a natural crystallite alignment degree of the natural silk fiber, and wherein a comprehensive orientation function <ƒₑ> associated with the higher crystallite alignment, is larger than a comprehensive orientation function <ƒₙ> associated with the natural crystallite alignment.

47. The enhanced protein material of claim 46, wherein the comprehensive orientation function <ƒₑ> is larger than 0.84.

48. (canceled)

49. The enhanced protein material of claim 1, wherein said enhanced protein material comprises at least one of the following properties: the enhanced protein material has substantially the same primary protein structure as the natural silk fiber, the enhanced protein material has a diameter substantially equal to a natural diameter of the natural silk fiber, and the enhanced protein material responds to a environmental conditions comprising temperature and humidity in a way that does not differ substantially from the natural silk fiber.

50-55. (canceled)

56. An enhanced silk fiber produced by a silkworm, wherein the enhanced silk fiber is not a regenerated silkworm-silk fiber, the enhanced silk fiber having
a length that is more than 10 m,
a yield stress that is more than 150 MPa,
a yield strain that is more than 1.8%
a breaking stress that is more than 505 MPa,
a breaking strain that is more than 17%,
a breaking energy that is more than 50 kJ/kg,
a comprehensive orientation function <ƒₑ> that is more than 0.89,
a diameter that is substantially equal to a diameter of a natural silkworm-silk fiber,
the enhanced silk fiber comprising a primary protein structure that is substantially equal to a protein structure of the natural silkworm-silk fiber,
the enhanced silk fiber responding to environmental conditions comprising a temperature and a humidity in a way that does not differ substantially from the natural silkworm-silk fiber,
the natural silkworm-silk fiber being extruded by the silkworm naturally in an absence of an intervention.

57-60. (canceled)

61. A method of formation of an enhanced protein material comprising the steps of:
a) taking an animal of an invertebrate species that is able to extrude a protein material,
b) applying a stimulus to the animal, wherein the stimulus excludes reeling,
the stimulus enhancing a mechanical performance of a natural protein material,
the natural protein material being the protein material extruded by the animal in an absence of the stimulus,
and
c) collecting the enhanced protein material extruded by the animal.

62. The method of claim 61, wherein the step (b) further comprises applying the stimulus while the enhanced protein material is being extruded by the animal.

63. The method of claim 61, wherein the step (b) further comprises applying the stimulus upon a beginning of an extrusion of the enhanced protein material, and maintaining the stimulus until a completion of the extrusion.

64. The method of claim 61, further comprising a step (d) comprising post-stretching the enhanced protein material after collecting the enhanced protein material extruded by the animal.

65. The method of claim 61, wherein the stimulus is one of: an electromagnetic field, a radiation field, an optical stimulus and an acoustic stimulus.

66. The method of claim 65, wherein the electromagnetic field is an electric field.

67. The method of claim 66, wherein the invertebrate species is a silkworm species and the animal is a silkworm selected from a group comprising Bombyx mori, Philosamia Cynthia and Telea Polyphemus.

68. (canceled)

69. The method of claim 67 wherein the step (a) further comprises
preparing an apparatus for applying the alternating electric field to silkworms,
the apparatus comprising a container, the container being suitably partitioned for placing the silkworms such that the electric field remains unaffected by the container, and placing the silkworms in the partitioned container.

70. The method of claim 69 wherein the step (b) further comprises the steps of:
activating the electric field when the silkworms begin to spin, thereby
bringing about an enhancement of a mechanical performance of the natural silk fiber,
the mechanical performance comprising one or more of: a yield stress, a yield strain, a breaking stress, a breaking strain, a breaking energy, and an elastic modulus.

71. The method of claim 70 wherein the electric field is an alternating electric field with at least one of the following properties: a peak-to-peak strength in a range 0-2000 V/cm or a frequency in a range of 0-2 MHz.

72-81. (canceled)

82. The method of claim 61, wherein the method comprises the steps of:
a) taking one or more silkworms,
b) preparing an apparatus for applying an alternating electric field to the one or more silkworms,
the alternating electric field having an alternating electric field strength is in a range 0-600 V/cm and an alternating electric field frequency is in a range 0-5000 Hz,
the apparatus comprising a container,
the container being suitably partitioned for placing the one or more silk worms such that the alternating electric field remains unaffected by the container,
c) placing the one or more silkworms in the partitioned container,
d) activating the alternating electric field when the one or more silkworms begin a spinning process, thereby bringing about an enhancement of a mechanical performance of a natural silk fiber, the natural silk fiber being extruded by the one or more silk worms in an absence of the alternating electric field,
the mechanical performance comprising one or more of: a yield stress, a yield strain, breaking stress, a breaking strain, a breaking energy and an elastic modulus,
e) maintaining the alternating electric field until completion of the spinning process, and
f) collecting the enhanced silk fiber extruded by the one or more silkworms.

83-88. (canceled)
